(19) United States
(12) Patent Application Publication
(10) Pub. No.: US 2021/0196404 A1
(43) Pub. Date: Jul. 1, 2021

(54) IMPLEMENTATION METHOD FOR OPERATING A SURGICAL INSTRUMENT USING SMART SURGICAL GLASSES

(71) Applicant: TAIWAN MAIN ORTHOPAEDIC BIOTECHNOLOGY CO., LTD., Taichung City (TW)

(72) Inventor: Min-Liang Wang, Taichung City (TW)

(21) Appl. No.: 16/729,694

(22) Filed: Dec. 30, 2019

(51) Int. Cl. A61B 34/00; G06F 3/0482; G06T 19/00

(52) U.S. Cl. CPC A61B 34/25; A61B 2090/3762; G06T 19/006; G06F 3/0482

(57) ABSTRACT

An implementation method for operating a surgical instrument using smart surgical glasses for a surgeon to perform an operation on an surgical site with at least one surgical instrument mainly comprises the steps of: the surgeon wearing a smart surgical glasses; using the sensors to sense the position of the surgical instrument held by the surgeon and the position of the surgical site; displaying a picture on a display of the smart surgical glasses, the picture comprising a plurality of sub-blocks; combining the medical image block, the instrument block and the surgical site block to form a mixed reality image; displaying an image of the surgical planning block in the mixed reality image; and the surgeon operating the surgical instrument according to the image of the surgical planning block.

### BACKGROUND OF THE INVENTION

### 1. Field of the Invention

The present invention relates to a surgical implementation method, and more particularly to an implementation method for operating a surgical instrument using smart surgical glasses.

### 2. Description of the Related Art

In recent years, with the development of new health care technology, computer-assisted surgery has increased significantly. Since the accuracy of surgical instruments and imaging technology has improved, doctors not only can enhance the quality of their surgery, but also can minimize patient wounds. Generally, a computer-assisted surgery consists of four parts: acquiring images from a patient, image analysis and processing, pre-diagnosis and surgical planning simulation, and finally receiving the surgery guidance for the patient. Computer-assisted surgery currently is divided into the following steps: first, using tomography images, including computerized tomography (CT), magnetic resonance imaging (MRI), X ray, nuclear medicine imaging, reconstructed 3D models (non-real-time image), and second: using the mobile C-arm X-ray machine, ultrasound imaging or endoscopy systems in the operating room as an auxiliary guide (real-time image) and a non-image-based guidance system.

Clinical application of image guided surgical systems, including spinal surgery guide (e.g., pedicle screw fixation, removal of damaged sites, removal of lumps, and disposing electrode to a fixed depth for epilepsy patients); head lesion surgery (e.g., treatment of meningioma, craniopharyngioma, chondrosarcoma, and other lesions in the cranial portion); tumor resection tissue sections; treatment of Parkinson's disease; treatment of huibu brain stereotaxic of psychiatric disorders; audition functional sinus surgery; neurovascular surgical repair and ventricular bypass surgery and ventricular shunt replacement surgery. This system can also be used for the hip and knee surgery, such as total knee arthroplasty, total hip replacement surgery, and anterior cruciate ligament reconstruction.

Operation must be combined with image guide, electronic, machinery, and other techniques, so the orientation of the surgical instrument projected onto the image may assist a physician to grasp the relative orientation between the device and the patient and to achieve the purpose of navigation. During the operation, the doctor puts a mark on the patient's surgical position, and then allows the patient to undergo a computerized tomography or magnetic resonance imaging examination. The image of computerized tomography or magnetic resonance image is reconstructed in the computer to form the three-dimensional position near the surgical site, and the location of the anomaly and normal functional area are indicated. At the time of surgery, the surgical site of the patient and the surgical instruments—need mounting marks, and then infrared camera (ultrasound or the like) can label the location and relative positions of the surgical site and the surgical instrument simultaneous to create space surgery relationship according to these infrared signals reflected from the mounting mark. In addition, the surgeon may use the head or overhead display through the eyepiece to see the image reorganization.

Augmented Reality (Augmented Reality, AR) and Mixed Reality (Mixed Reality, MR) are generally used to display virtual information on the real image of the patient. Particularly in minimally invasive surgery using the endoscope in the past, the overlay of images is performed in the augmented and mixed reality images. This way can not be directly observed by the camera, but now the image can be seen prior to surgery. Augmented and mixed reality assist the surgeon to see through the patient's body part, so that prior to the surgical site visits, vital structures thereof can be effectively positioned without confirming the position beforehand by performing the operation. Currently, augmented and mixed reality technology seems to be the most promising research, which helps guide the surgeon and perform supervision robotic surgery.

In view of the above problems, it is desired to provide an implementation method for operating a surgical instrument using smart surgical glasses for a surgeon.

### BRIEF SUMMARY OF THE INVENTION

An object of the present invention is to provide an implementation method for operating a surgical instrument using smart surgical glasses for a surgeon that can rapidly establish augmented and mixed reality of a surgical instrument for the application of augmented and mixed reality computer assisted glasses for surgical operation.

An implementation method for operating a surgical instrument using smart surgical glasses for a surgeon to perform an operation on an surgical site with at least one surgical instrument comprises the following steps of:

Step 1: the surgeon wearing a smart surgical glasses, wherein the smart surgical glasses are provided with a plurality of sensors;

Step 2: using the sensors to sense the position of the surgical instrument held by the surgeon and the position of the surgical site to obtain a true position image of the instrument and a true position image of the surgical site;

Step 3: displaying a picture on a display of the smart surgical glasses, the picture comprising a plurality of sub-blocks, the sub-blocks comprising: a medical image block, a surgical planning block, a block for displaying the true position image of the instrument and a block for displaying the true position image of the surgical site;

Step 4: combining the medical image block, the instrument block and the surgical site block to form a mixed reality image and then enlarging the mixed reality image gradually to fill the entire display of the smart surgical glasses, wherein the image of the medical image block presents a three-dimensional virtual image;

Step 5: displaying an image of the surgical planning block in the mixed reality image;

Step 6: the surgeon operating the surgical instrument to perform the operation on the surgical site according to the image of the surgical planning block.

According to one aspect of the present invention, the sensors are cameras.

According to one aspect of the present invention, the step 2 further comprises the step of performing an immediate correction on the true position of the surgical instrument.

According to one aspect of the present invention, the step 2 further comprises the step of performing an immediate correction on the true position of the surgical site.

According to one aspect of the present invention, in step 3, the sub-blocks included in the picture can be clicked and enlarged into the entire picture, and can be reduced to the sub-blocks.

According to one aspect of the present invention, in step 3, a menu appears when pressing and holding on any one of the blocks of the display.

According to one aspect of the present invention, in step 3, a menu appears, including a list of: computerized tomography (CT), magnetic resonance imaging (MRI), X-ray, positron image and nuclear medicine image when long pressing on the medical image block of the display.

According to one aspect of the present invention, in step 3, a menu appears, including a list of a plurality of different surgical planning processes when pressing and holding on the surgical planning block of the display.

According to one aspect of the present invention, in step 4, combining the different images is performed by an image overlay software.

According to one aspect of the present invention, the smart surgical glasses are provided with a wireless transmission module for outputting the picture on the display of the smart surgical glasses to a display system in the vicinity.

Using the implementation method according to the present invention, the doctor/physician/surgeon can tell specifically and instantly whether the positional relationship between the surgical instrument and the patient's surgical site conforms to the surgical planning, and specifically increases the accuracy of the surgical instrument on the surgical site of the patient.

### BRIEF DESCRIPTION OF THE DRAWINGS

FIG. 1 is a smart surgical glasses according to the present invention.

FIG. 2 is a schematic diagram of the picture on the display of the smart surgical glasses.

FIG. 3 is a schematic diagram of the picture on a display of the smart surgical glasses showing a mixed reality image.

FIG. 4 is a procedure flowchart of the implementation method for operating a surgical instrument using smart surgical glasses for a surgeon according to the present invention.

FIG. 5 is a schematic diagram of the picture on the display which one of the blocks shows a menu.

### DETAILED DESCRIPTION OF THE INVENTION

While the present invention can be expressed in different forms of embodiment, the drawings and the following description is merely of preferred embodiments of the present invention. It is known that examples of the present invention are not intended to limit the invention to the illustrated and/or described in a particular embodiment.

The present invention provides an implementation method for operating a surgical instrument that can be applied to computer-assisted glasses with enhanced authenticity for surgery (Augmented Reality Computer Assisted Glasses for Surgical Operation). The enhanced authenticity can be seen as a mixture of virtual and real-world space that synchronizes patient information.

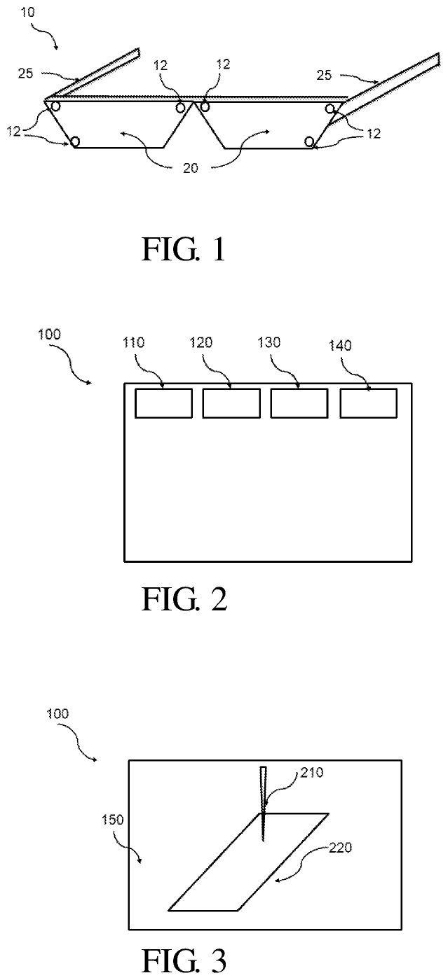

FIG. 1 is a smart surgical glasses of the present invention. The smart surgical glasses 10 comprises a display 20 and a pair of arms 25. The smart surgical glasses 10 are provided with a plurality of sensors 12.

The smart surgical glasses 10 has a surgical planning function, a sensing function, an image coincidence function and a real-time tracking function of surgical instruments.

The smart surgical glasses 10 comprises a processing module to process the above functions. The processing module can be a microprocessor or a center processing unit (CPU). The processing module is embedded in the smart surgical glasses 10. Or, the processing module is set on another computer unit, such as a computer, a workstation and a processing broad etc, and provides signals of the above functions to the smart surgical glasses 10.

The surgical planning function is used for the construction of preoperative surgical plan information for the doctor/physician/surgeon. The preoperative surgical plan information includes the two-dimensional medical images and three-dimensional medical images of the surgical site of the patient.

The sensing function is used for capturing the patient's surgical site and a real-time dynamic image of a surgical instrument movement track.

The sensing function is built on the smart surgical glasses 10 worn by the doctor/physician/surgeon. The sensing function uses one or more sensors 12 of the same or different functions, including but not limited to IR camera, Color camera, Depth camera and CCD camera. For example, two cameras are used in an embodiment, one is a CCD camera, and the other is the IR camera.

The image coincidence function is used to import a three-dimensional medical image of the surgical planning function and displays the three-dimensional medical image on the display 20 of the smart surgical glasses 10. The image coincidence function adjusts an angle of the three-dimensional medical image on the display according to a focal length and a viewing angle of the doctor's eyes so that the three-dimensional medical images is coincident with a real field of view of a doctor viewing the surgical site of the patient. In the embodiment of the present invention, a method for enhancing the image reality (see e.g. Republic of China Invention Patent Application No. 105134457 and United States Patent Publication No. 2019-0216572) is adopted to obtain the correct position of the doctor's eye on the marked point of the surgical site of the patient so as to adjust the error between the three-dimensional medical image and the marked point.

The real-time tracking function of surgical instruments, through the real-time dynamic image of a surgical instrument movement track captured by the sensors 12 of the smart surgical glasses 10, is used to real-time calculate a position difference between the tail point of the surgical instrument and the entrance point of the surgical planning function.

FIG. 2 is a schematic diagram of the picture 100 on the display 20 of the smart surgical glasses 10. The picture 100 comprises a plurality of sub-blocks, including: a medical image block 110, a surgical planning block 120, a block 130 for displaying the true position image of the instrument and a block 140 for displaying the true position image of the surgical site.

FIG. 3 is a schematic diagram of the picture 100 on a display 20 of the smart surgical glasses 10 showing a mixed reality image 150. The mixed reality image 150 enlarges the medical image block 110 gradually to fill the entire display 20 of the smart surgical glasses 10. In this embodiment, the mixed reality image 150 displays the instrument 210 and the surgical site 220 in the three-dimensional medical image.

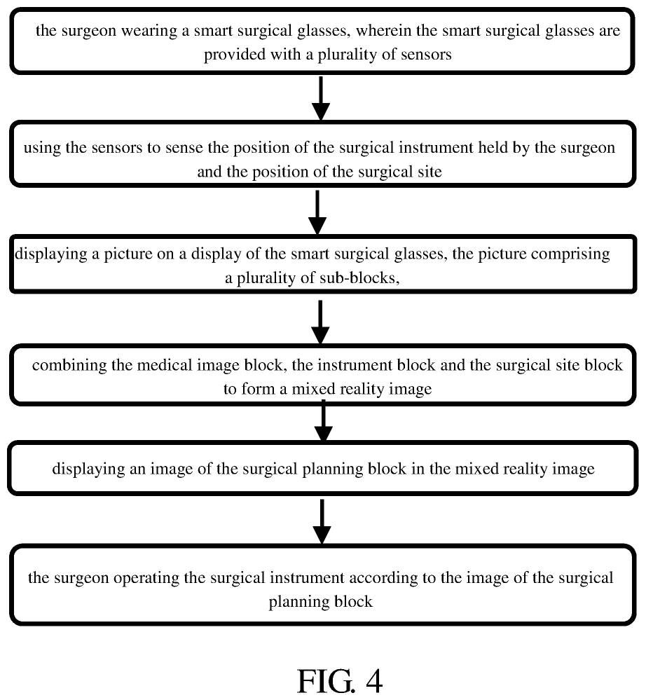

FIG. 4 is a procedure flowchart of an implementation method for operating a surgical instrument using smart surgical glasses for a surgeon of the present invention.

The disclosed implementation method is used for operating a surgical instrument using smart surgical glasses for a surgeon to perform an operation on a surgical site with at least one surgical instrument. The implementation method comprises the following steps of:

Step 1: the surgeon wearing a smart surgical glasses 10, wherein the smart surgical glasses 10 are provided with a plurality of sensors 12;

Step 2: using the sensors 12 to sense the position of the surgical instrument held by the surgeon and the position of the surgical site to obtain a true position image of the instrument and a true position image of the surgical site;

Step 3: displaying a picture 100 on a display 20 of the smart surgical glasses 10, the picture 100 comprising a plurality of sub-blocks, the sub-blocks comprising: a medical image block 110, a surgical planning block 120, a block 130 for displaying the true position image of the instrument and a block 140 for displaying the true position image of the surgical site;

Step 4: combining the medical image block 110, the instrument block 130 and the surgical site block 140 to form a mixed reality image 150 and then enlarging the mixed reality image 150 gradually to fill the entire display 20 of the smart surgical glasses 10, wherein the image of the medical image block 110 presents a three-dimensional virtual image;

Step 5: displaying an image of the surgical planning block 120 in the mixed reality image 150;

Step 6: the surgeon operating the surgical instrument to perform the operation on the surgical site according to the image of the surgical planning block.

Through the smart surgical glasses, a doctor/physician/surgeon can instantly obtain a real scene of the surgical site of the patient, a three-dimensional medical image that is coincident with a surgical site of the patient, and a real-time positional relationship between the surgical instrument and the surgical site of the patient.

In addition, through the mixed reality technology, the entrance guiding interface, the angle guiding interface, and the depth guiding interface are presented in real time in a real scene viewed through the smart surgical glasses.

The step 2 further comprises the following step of performing an immediate correction on the true position of the surgical instrument.

The step 2 further comprises the following step of performing an immediate correction on the position of the surgical site.

For the immediate correction, the translation matrix formula is used to obtain from the positional characteristics of the center point of each geometric pattern of the position of the surgical instrument and the position of the surgical site detected by the sensors 12. The translation matrix formula is then processed in the processing module of the smart surgical glasses 10 using a function library and then performed by a mathematical operation. The translation matrix formula is from a three-dimensional model of the component library constructed. The function library uses OOOPDS rendering core algorithm, to construct the bounding box using the three-dimensional model component library; to implement collision detection; to calculate component libraries, to implement force feedback and serial communication using the data communication component library function console such as 802.11g, TCP/IP or RS232, etc. In an embodiment, the function library is a OOOPDS library written in C/C++ language.

In step 3, the sub-blocks included in the picture can be clicked and enlarged into the entire picture, and can be reduced to the sub-blocks.

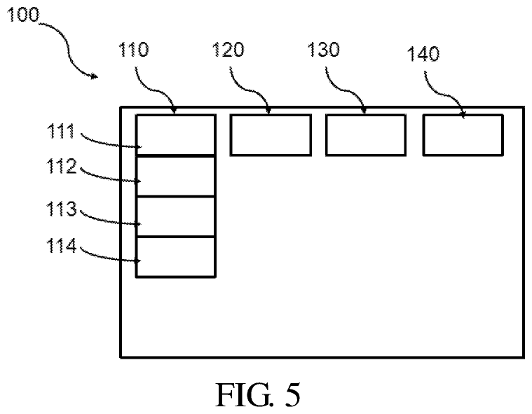

In step 3, a menu appears when pressing and holding on any one of the blocks of the display. FIG. 5 is a schematic diagram of the picture 100 on the display 20 with one of the sub-blocks showing a menu. For example, when pressing and holding on the medical image block 110 of the display 20, a menu appears, including a list of: computerized tomography (CT) 111, magnetic resonance imaging (MRI) 112, X-ray, positron image 113 and nuclear medicine image 114. In another embodiment, when pressing and holding on the surgical planning block of the display in the step 3, a menu appears, including a list of boa plurality of different surgical planning processes.

In this invention, the smart surgical glasses 10 has a surgical planning function, a sensing function, an image coincidence function and a real-time tracking function of surgical instruments. In step 4, combining the different images is performed by an image overlay software by using the image coincidence function of the smart surgical glasses 10. The image overlay software is operable to create and transmit laboratory prescriptions, such as digital models of anatomical features, to an on-site or off-site laboratory for use in fabricating a prosthetic (e.g., partial dentures, implant abutments, orthodontic appliances, and the like), surgical guides, or the like OOOPDS software.

The smart surgical glasses are provided with a wireless transmission module for outputting the picture on the display of the smart surgical glasses to a display system in the vicinity.

The processing module processes images and data, and communicates images and data via wired or wireless connections. For example, the image overlay software can be used by the medical clinician to manipulate, convert, and overlay images collected by the surgical site image required to be used in other surgical procedures. Although different machines may produce images in different formats, it is desirable that the image overlay software be capable of converting one or more image formats into another one or more different formats, so that the images collected by different devices can be displayed together in an overlying fashion. Thus, the image overlay software is configured to access, display, convert, and manipulate a new spatial variation image by combining the spatial variation image with the image of the surgical site to be used in other surgical procedures in various formats including, for example, DICOM images, CAD images, STL images, or the like. The image overlay software permits a clinician to review digital images, visualize virtual models and create images overlays on a display of the smart surgical glasses worn by a surgeon.

The doctor/physician/surgeon, by means of the smart surgical glasses 10, defines the working depth for the entire optical system on the smart surgical glasses 10 and, by moving his head, automatically, illuminates the operating area by pointing the headlight according to head movement. Thus the image which is returned from the surgical site is always directed along the same line as the illumination pattern. The surgeon, by moving his head, automatically aims the headlight, and the eyes of the surgeon perceive the area illuminated by the beam which then, based on the orientation of the optical system on the smart surgical glasses, produces an image which essentially is completely indicative of exactly what the surgeon is seeing and at the same magnification.

With the implementation method according to the present invention, the doctor/physician/surgeon can tell specifically and instantly whether the positional relationship between the surgical instrument and the patient's surgical site conforms to the surgical planning, and specifically increases the accuracy of the surgical instrument on the surgical site of the patient.

While the invention has been disclosed in the foregoing preferred embodiments, they are not intended to limit the present invention, and one skilled in the art may make various changes or modifications without departing from the spirit and scope of the present disclosure. Therefore the scope of the present invention is best defined by the appended claims.

What is claim is:

1. An implementation method for operating a surgical instrument using smart surgical glasses for a surgeon to perform an operation on a surgical site with at least one surgical instrument, the implementation method comprising the following steps of:
Step 1: the surgeon wearing a smart surgical glasses, wherein the smart surgical glasses are provided with a plurality of sensors;
Step 2: using the sensors to sense the position of the surgical instrument held by the surgeon and the position of the surgical site to obtain a true position image of the instrument and a true position image of the surgical site;
Step 3: displaying a picture on a display of the smart surgical glasses, the picture comprising a plurality of sub-blocks, the sub-blocks comprising a medical image block, a surgical planning block, a block for displaying the true position image of the instrument, and a block for displaying the true position image of the surgical site;
Step 4: combining the medical image block, the instrument block and the surgical site block to form a mixed reality image and then enlarging the mixed reality image gradually to entirely cover the display of the smart surgical glasses, wherein the image of the medical image block is presented as a three-dimensional virtual image;
Step 5: displaying an image of the surgical planning block in the mixed reality image; and
Step 6: the surgeon operating the surgical instrument to perform the operation on the surgical site according to the image of the surgical planning block.

2. The implementation method according to claim 1, wherein the sensors are cameras.

3. The implementation method according to claim 1, wherein the step 2 further comprises performing an immediate correction on the true position of the surgical instrument.

4. The implementation method according to claim 1, wherein the step 2 further comprises performing an immediate correction on the true position of the surgical site.

5. The implementation method according to claim 1, wherein in step 3, the sub-blocks included in the picture may be clicked and enlarged into the entire picture, and clicked to be reduced to the sub-blocks.

6. The implementation method according to claim 1, wherein in step 3, a menu appears when pressing and holding on any one of the blocks of the display.

7. The implementation method according to claim 1, wherein in step 3, a menu appears, including a list of: computerized tomography (CT), magnetic resonance imaging (MRI), X-ray, positron image and nuclear medicine image when pressing and holding on the medical image block of the display.

8. The implementation method according to claim 1, wherein in step 3, a menu appears, including a list of different surgical planning processes when pressing and holding on the surgical planning block of the display.

9. The implementation method according to claim 1, wherein in step 4, combining the different images is performed by an image overlay software.

10. The implementation method according to claim 1, wherein the smart surgical glasses are provided with a wireless transmission module for outputting the picture on the display of the smart surgical glasses to a display system in a vicinity.
